(19) United States
(12) Patent Application Publication
(10) Pub. No.: US 2017/0023481 A1
(43) Pub. Date: Jan. 26, 2017

(54) HYDROGEN GAS INSPECTION METHOD

(71) Applicant: Panasonic Intellecutal Property Management Co., Ltd., Osaka (JP)

(72) Inventors: YOSHIHISA NAGASAKI, Osaka (JP); NOBUYASU SUZUKI, Osaka (JP); YUKIHIKO SUGIO, Osaka (JP)

(21) Appl. No.: 15/194,059

(22) Filed: Jun. 27, 2016

(30) Foreign Application Priority Data
Jul. 22, 2015 (JP) 2015-144981

(51) Int. Cl. G01N 21/65

(52) U.S. Cl. CPC G01N 21/65; G01N 2201/0612; G01N 33/005

(57) ABSTRACT

A hydrogen gas inspection method includes: converting first light having a first wavelength to second light having a second wavelength longer than the first wavelength by using a phosphor, the first light being emitted from a semiconductor light emitting device; irradiating a space to be inspected with the second light; and determining whether hydrogen gas is present in the space utilizing Raman scattered light generated by the hydrogen gas irradiated with the second light.

### BACKGROUND

1. Technical Field

The present disclosure relates to a hydrogen gas inspection method and a hydrogen gas inspection device.

2. Description of the Related Art

Conventionally, a hydrogen sensor such as a contact burning-type sensor or a semiconductor-type sensor has been proposed as means for detecting leakage of hydrogen gas in a hydrogen refueling station or a fuel cell system. Detection of hydrogen gas using a contact burning-type sensor or a semiconductor-type sensor requires that hydrogen gas make contact with a sensor unit. Therefore, there is a problem that it is not easy to specify a leaking part. Furthermore, there is a problem that hydrogen gas does not reach the sensor unit depending on a place of installation of the sensor or a direction of diffusion of the gas and therefore sufficient detection is impossible. As for flammable gas such as methane and propane other than hydrogen gas, there are methods (e.g., infrared absorption type) that make it possible to detect gas to be inspected without requiring direct contact between the gas to be inspected and a sensor unit.

As a method for detecting hydrogen gas without direct contact between hydrogen gas and a sensor unit, for example, Japanese Patent No. 3783019 describes a method for detecting hydrogen gas by emitting laser light into a target space and then collecting Raman scattered light that is the laser light scattered by the hydrogen gas. The method for detecting hydrogen gas by using Raman scattered light is a method utilizing a phenomenon in which when hydrogen gas is irradiated with light of any wavelength, Raman scattered light having a wavelength shifted from the wavelength of the irradiation light by energy corresponding to vibrational energy or rotational energy of hydrogen molecules is generated.

### SUMMARY

One non-limiting and exemplary embodiment provides a hydrogen gas inspection method that safely inspect the presence or absence and/or the concentration of hydrogen gas, which is colorless and odorless, in a non-contact manner.

In one general aspect, the techniques disclosed here feature a hydrogen gas inspection method including: a hydrogen gas inspection method includes: converting first light having a first wavelength to second light having a second wavelength longer than the first wavelength by using a phosphor, the first light being emitted from a semiconductor light emitting device; irradiating a space to be inspected with the second light; and determining whether hydrogen gas is present in the space utilizing Raman scattered light generated by the hydrogen gas irradiated with the second light.

According to a hydrogen gas inspection method of the present disclosure, it is possible to safely inspect the presence or absence and/or the concentration of hydrogen gas, which is colorless and odorless, in a non-contact manner.

It should be noted that general or specific embodiments may be implemented as a device, a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an outline configuration of a hydrogen gas inspection device using a laser-excited phosphor transmission type light emission light source according to First Embodiment of the present disclosure;

FIG. 2 is a diagram illustrating an outline configuration of a hydrogen gas inspection device using a laser-excited phosphor reflection type light emission light source according to First Embodiment of the present disclosure;

FIG. 3 is a diagram illustrating an outline configuration of a hydrogen gas inspection device according to Second Embodiment of the present disclosure;

FIG. 4 is a diagram illustrating an outline configuration of a hydrogen gas inspection device according to Third Embodiment of the present disclosure;

FIG. 5 illustrates an example of a phosphor emission spectrum and a laser spectrum according to the present disclosure;

FIG. 6 illustrates spectra of Rayleigh scattered light, Raman scattered light scattered by hydrogen gas, Raman scattered light scattered by oxygen gas, and Raman scattered light scattered by nitrogen gas obtained in a case where a wavelength region of irradiation light is not less than 500 nm and not more than 550 nm according to the present disclosure; and

FIG. 7 illustrates spectra of Rayleigh scattered light, Raman scattered light scattered by hydrogen gas, Raman scattered light scattered by oxygen gas, and Raman scattered light scattered by nitrogen gas obtained in a case where a wavelength region of irradiation light is not less than 470 nm and not more than 520 nm according to the present disclosure.

### DETAILED DESCRIPTION

### Underlying Knowledge Forming Basis of the Present Disclosure

The method for detecting Raman scattered light that is laser light scattered by hydrogen gas has a problem that accuracy of inspection declines due to influence of ambient light since Raman scattered light is much weaker than Rayleigh scattered light having the same wavelength as the irradiation laser light. Furthermore, since the intensity of Raman scattered light is proportionate to the concentration of hydrogen gas, there is a problem that it is difficult to detect especially hydrogen gas of low concentration. It is therefore necessary to increase the intensity of irradiation laser light in order to improve accuracy of inspection. However, the intensity of laser light that can be emitted is legally regulated in an area except for a specific laser controlled area in order to prevent damage on eyes or skin. Therefore, there is a problem that Raman scattered light that is strong enough to detect hydrogen gas cannot be obtained in a case where the intensity of laser light is within a safety regulation. In view of the above problems, the inventors of the present invention diligently conducted research to provide a hydrogen gas inspection method and a hydrogen gas inspection device that safely and precisely analyze hydrogen gas, which is colorless and odorless, in a non-contact manner.

The present disclosure relates to a hydrogen gas inspection method and a hydrogen gas inspection device that safely and speedily conduct detection and/or quantitative measurement of hydrogen gas, which is colorless and odorless. The hydrogen gas detection method according to the present disclosure uses a laser-excited phosphor light emission light source that excites a phosphor by using laser light and radiates, as light for causing the Raman scattering phenomenon, light whose wavelength has been converted by the phosphor instead of coherent laser light whose intensity should be within the safety regulation.

Unlike laser light, the light radiated from the laser-excited phosphor light emission light source has uneven phases due to wavelength conversion and scattering by the phosphor and is not amplified due to interference and is therefore exempt from the safety regulation for laser light. Meanwhile, coherence (even phases) of laser light is not a required property of light that causes the Raman scattering phenomenon.

Furthermore, since the laser-excited phosphor light emission light source allows a luminous point to be much smaller than a light source such as a halogen lamp or a discharge lamp, the intensity of light at an irradiation position can be strengthened to an intensity equivalent to the intensity of laser light in a case where the laser-excited phosphor light emission light source is used as a light source for causing Raman scattering.

The intensity of Raman scattered light of hydrogen gas induced by delivering light emitted from the laser-excited phosphor light emission light source into a space to be inspected by using a lens or the like becomes higher as the intensity of the emitted light becomes higher, and in a case where the Raman scattered light is detected by using an optical sensor, a high S/N ratio can be obtained by blocking Rayleigh scattered light and Raman scattered light and fluorescence scattered by gas other than hydrogen gas by using an optical bandpass filter.

In the laser-excited phosphor light emission light source, any wavelength can be selected as a wavelength of emitted light by selecting the type of a used phosphor. Therefore, the presence of absence of hydrogen gas can be visually determined in a case where a phosphor that makes Raman scattered light scattered by hydrogen gas visible is selected as the used phosphor.

Furthermore, Rayleigh scattered light and Raman scattered light can be easily separated from each other by narrowing a wavelength region of light delivered from the phosphor of the laser-excited phosphor light emission light source into a space to be inspected by using an optical bandpass filter.

There are four types of Raman scattered light scattered by hydrogen gas, i.e., vibrational Stokes Raman scattered light, vibrational anti-Stokes Raman scattered light, rotational Stokes Raman scattered light, and rotational anti-Stokes Raman scattered light. It is only necessary that the hydrogen gas detection method according to the present disclosure detect at least one of the four types of Raman scattered light, but it is desirable that vibrational Stokes Raman scattered light be detected from the perspective of the intensity of Raman scattered light and a shift width of energy from Rayleigh scattered light.

The present disclosure is described in detail below by presenting specific embodiments. Needless to say, however, the present disclosure is not limited to these embodiments and can be changed as appropriate within the technical scope of the present disclosure.

A hydrogen gas inspection method according to an aspect of the present disclosure includes: converting first light having a first wavelength to second light having a second wavelength longer than the first wavelength by using a phosphor, the first light being emitted from a semiconductor light emitting device; irradiating a space to be inspected with the second light; and determining whether hydrogen gas is present in the space utilizing Raman scattered light generated by the hydrogen gas irradiated with the second light. The Raman scattered light may be detected from scattered light generated in the space to be inspected. The hydrogen gas inspection method may further include determining a concentration of the hydrogen gas in the space to be inspected.

The semiconductor light-emitting device may be a laser diode having a light emission peak wavelength of 360 nm to 500 nm. Note that in a case where there are a plurality of peaks, the “peak wavelength” in the present disclosure refers to a wavelength of a maximum peak.

A light emission peak wavelength of the second light may be 380 nm to 600 nm.

The space to be inspected may be irradiated with the second light via an optical bandpass filter, and a full width at half maximum of the second light that passes through the optical bandpass filter may be 10 nm to 100 nm.

An optical bandpass filter extracts the Raman scattered light from scattered light generated in the space to be inspected. An optical sensor may be used to detect the Raman scattered light.

the Raman scattered light may be visually checked by human eyes.

A spectrometer may extract the Raman scattered light from scattered light generated in the space to be inspected. An optical sensor may be used to detect the Raman scattered light

Another aspect of the present disclosure is a hydrogen gas inspection device comprising: a semiconductor light-emitting device that emits first light having a first wavelength; a phosphor that converts the first light to second light having a second wavelength longer than the first wavelength and irradiates a space to be inspected with the second light; and a light detection device that determines whether hydrogen gas is present in the space utilizing Raman scattered light generated by the hydrogen gas irradiated with the second light.

Embodiments are specifically described below with reference to the drawings. Each of the embodiments described below is a general or specific example of the present disclosure. Numerical values, shapes, materials, constituent elements, positions and connection forms of the constituent elements, steps, order of the steps, and the like described in the embodiments below are examples and do not limit the present disclosure. Among the constituent elements in the embodiments below, constituent elements that are not described in the independent claims that show highest concepts of the present disclosure are described as optional constituent elements. Furthermore, constituent elements that are identical or similar are given identical reference signs, and overlapping description thereof is sometimes omitted.

### First Embodiment

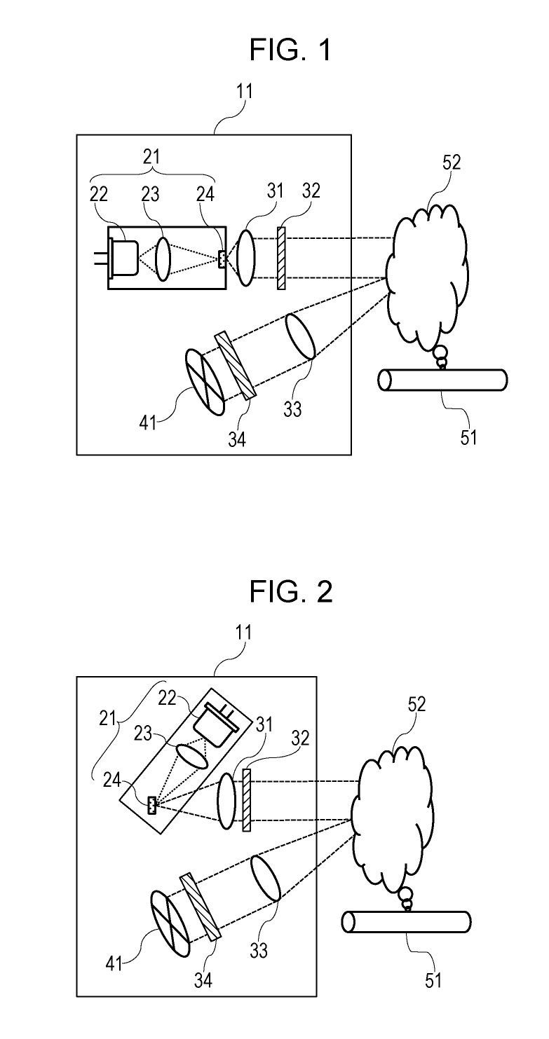

FIGS. 1 and 2 each illustrate an outline configuration of a device that inspects the presence or absence of hydrogen gas and concentration of hydrogen gas according to First Embodiment of the present disclosure. An inspection device 11 includes a light source device 21, a lens 31 for irradiation, an optical bandpass filter 32 for irradiation light, a lens 33 for light reception, an optical bandpass filter 34 for light reception, and a light detection device 41. The light source device 21 includes a semiconductor light-emitting device 22, a light collecting lens 23, and a phosphor element 24.

The semiconductor light-emitting device 22 may be a laser diode having a light emission peak wavelength of not less than 360 nm and not more than 500 nm. This allows light emitted from the semiconductor light-emitting device 22 to be efficiently converted by a phosphor and be focused into a small region. It is therefore possible to increase light use efficiency. A peak wavelength of the light emitted from the semiconductor light-emitting device 22 can be selected as appropriate in view of a wavelength range and/or conversion efficiency of the wavelength-converted light obtained when the light is combined with the used phosphor material. In the present embodiment, a laser diode having a light emission wavelength of 445 nm is used as the semiconductor light-emitting device 22 so that conversion efficiency of (Y,Ga)₃Al₅O₁₂:Ce³⁺ composition that is the used phosphor material is maximized.

The semiconductor light-emitting device 22 may be a single device or may be a plurality of devices. In a case where the semiconductor light-emitting device 22 is a plurality of devices, the phosphor element 24 is irradiated by a plurality of beams of light that have been coupled, for example, by using a plurality of light collecting lenses or by using an optical fiber. The power of laser with which the phosphor element 24 is irradiated is not restricted by the safety standard for laser light and the like and can be adjusted as appropriate in accordance with sensitivity of an optical detector or an environment in which the optical detector is used. The phosphor element 24 that converts light emitted from the semiconductor light-emitting device 22 may be a single crystal or ceramics constituted by a phosphor only or may be phosphor particles embedded in at least one of a matrix of an organic resin, a matrix of an organic-inorganic hybrid material, and a matrix of an inorganic material.

In FIG. 1, the phosphor element 24 is provided on a substrate (not illustrated) that allows light emitted from the semiconductor light-emitting device 22 to pass therethrough. The light emitted from the semiconductor light-emitting device 22 enters the substrate from a substrate surface side, and the wavelength of the light that has passed through the substrate is converted by a phosphor provided on the substrate. A dichroic mirror that allows light emitted from the semiconductor light-emitting device 22 to pass therethrough and reflects light whose wavelength has been converted by the phosphor element 24 may be provided on the substrate.

In FIG. 2, the wavelength of light emitted from the semiconductor light-emitting device 22 toward the phosphor element 24 is converted by the phosphor element 24, and then the light is output to the same side of the phosphor element 24. In this case, the substrate that supports the phosphor element 24 may be provided on a side of the phosphor opposite to the light emitted from the semiconductor light-emitting device 22 and the output light. Furthermore, in this case, the substrate may be one that reflects light. In FIG. 2, the phosphor element 24 may be provided on a substrate that absorbs light emitted from the semiconductor light-emitting device 22 and reflects light whose wavelength has been converted by the phosphor element 24.

A phosphor material used for the phosphor element 24 is not limited to a specific material composition, and a phosphor material of various kinds can be used, provided that the phosphor material is a material that can convert the wavelength of light emitted from the semiconductor light-emitting device 22. In the present embodiment, (YGa)₃Al₅O₁₂:Ce³⁺ composition is used as the phosphor material.

Vibrational Stokes Raman scattered light scattered by hydrogen gas is light having energy that is lower than irradiation light by 0.000416 nm⁻¹ which is vibrational energy intrinsic to hydrogen gas, and a relationship expressed by the following formula 1 is established between the irradiation light and the vibrational Stokes Raman scattered light (hereinafter referred to as Raman scattered light):

Raman scattered light [nm]=1/(1/irradiation light [nm]−0.000416 [nm⁻¹])

Accordingly, in a case where the wavelength of the irradiation light that induces vibrational Stokes Raman scattered light scattered by hydrogen gas is not less than 380 nm and not more than 600 nm, light with which a space 52 to be inspected is irradiated is visible, and the wavelength of the vibrational Stokes Raman scattered light scattered by hydrogen gas is not less than 508.9 nm and not more than 799.6 nm and is suitable for visual check or use of a visible region optical sensor. A peak wavelength of light whose wavelength has been converted by the phosphor element may be not less than 380 nm and not more than 600 nm.

The lens 31 is for delivering light emitted from the light source device 21 to the space 52 (e.g., the vicinity of a hydrogen gas pipe 51) to be inspected in which leakage of hydrogen gas is to be inspected. The lens 31 may be a lens unit made up of at least one lens.

The optical bandpass filter 32 plays a role of removing light emitted from the semiconductor light-emitting device 22 and part of light whose wavelength has been converted by the phosphor element 24 and may be a dielectric multi-layer type filter or may be an absorption type filter.

A wavelength region of light that has passes through the optical bandpass filter 32 may be not less than 380 nm and not more than 600 nm.

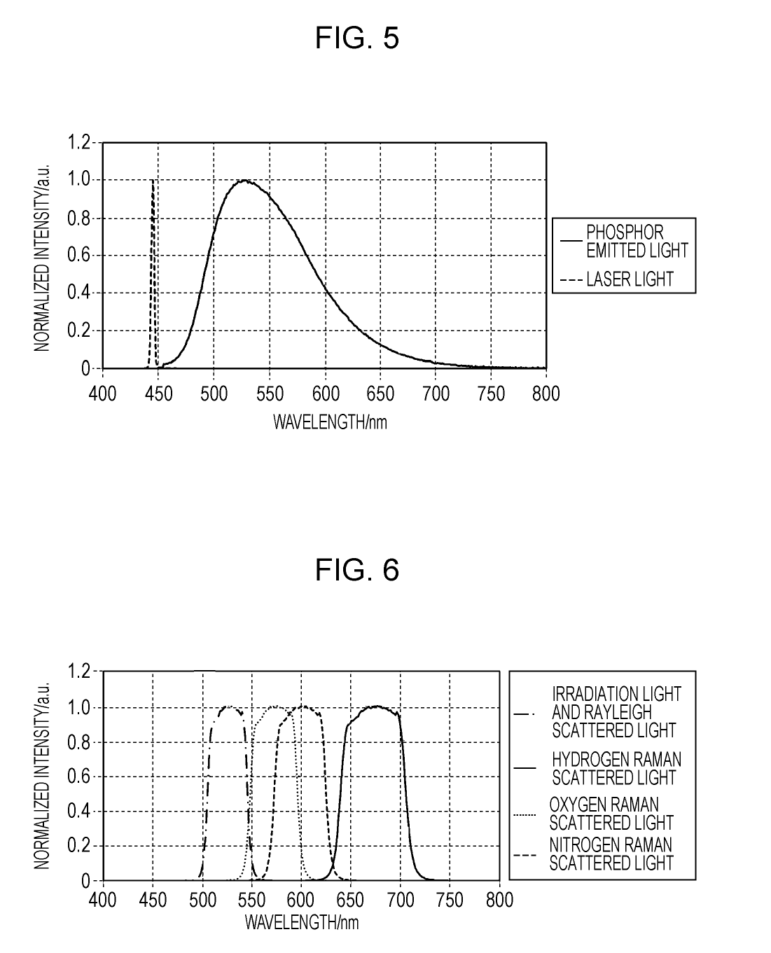

As illustrated in FIG. 5, the (Y,Ga)₃Al₅O₁₂:Ce³⁺ composition phosphor used in the present embodiment has a light emission distribution in a wide wavelength range from 470 nm to 750 nm. In the present embodiment, a wavelength distribution of light applied to the space 52 to be inspected can be selected by appropriately adjusting a wavelength region of light that passes through the optical bandpass filter 32 out of the light delivered from the phosphor. It is desirable that a full width at half maximum (FWHM) of a spectrum of light applied to the space 52 to be inspected be not more than 100 nm and not less than 10 nm. In a case where the FWHM of the irradiation light is not more than 100 nm, it is possible to easily separate Raman scattered light scattered by hydrogen gas and Raman scattered light scattered by oxygen gas or nitrogen gas in the atmosphere. Furthermore, in a case where the FWHM of the irradiation light is not less than 10 nm, it is possible to precisely detect Raman scattered light scattered by hydrogen gas.

FIG. 6 illustrates spectrum shapes of Rayleigh scattered light, Raman scattered light scattered by hydrogen gas, Raman scattered light scattered by oxygen gas, and Raman scattered light scattered by nitrogen gas in a case where the wavelength region of the irradiation light is not less than 500 nm and not more than 550 nm and the FWHM of the irradiation light is 40 nm. Intensities of the spectrum shapes have been normalized. As illustrated in FIG. 6, in a case where the wavelength region of light that has passed through the optical bandpass filter 32 is not less than 500 nm and not more than 550 nm, vibrational Stokes Raman scattered light scattered by hydrogen gas has a wavelength region of not less than 631.3 nm and not more than 713.2 nm and a central wavelength of 671.7 nm. Meanwhile, vibrational Stokes Raman scattered light scattered by nitrogen gas, which is a main component gas in the atmosphere, is not less than 566 nm and not more than 630.9 nm, and vibrational Stokes Raman scattered light scattered by oxygen gas, which is a main component gas in the atmosphere, is not less than 542.2 nm and not more than 601.5 nm. It is therefore possible to easily separate only the vibrational Stokes Raman scattered light scattered by hydrogen gas. The Raman scattered light generated by the hydrogen gas is detected from those scattered light generated in the space 52 to be inspected.

The lens 33 is for delivering light from the space 52 to be inspected to the light detection device 41 and may be a lens unit made up of at least one lens.

The optical bandpass filter 34 allows the Raman scattered light scattered by the hydrogen gas to pass therethrough and removes Rayleigh scattered light, Raman scattered light scattered by gas other than the hydrogen gas, and ambient light. In other words, the optical bandpass filter 34 extracts the Raman scattered light of hydrogen gas from scattered light generated in the space 52 to be inspected. The optical bandpass filter 34 may be a dielectric multi-layer type filter or may be an absorption type filter.

An optical sensor used for the light detection device 41 is not limited to an optical sensor of a specific type. The optical sensor is, for example, an avalanche photodiode or a photomultiplier tube. Furthermore, in a case where a CCD image sensor, a CMOS image sensor, or the like is used as the optical sensor, it is possible to grasp a hydrogen gas distribution as an image. The light detection device 41 may include a processing device (e.g., a microcomputer or a processor) that processes a signal from the optical sensor and a storage medium (e.g., a semiconductor memory or a hard disc) in which a processing program and processing data are stored. The processing device of the light detection device 41 detects Raman scattered light scattered by hydrogen gas, for example, by extracting data of a frequency component in a predetermined range from signal data supplied from the optical sensor and determines the presence or absence of the hydrogen gas and/or the concentration of the hydrogen gas on the basis of the detection result. In other words, whether hydrogen gas is present in the space 52 to be inspected is determined by utilizing Raman scattered light generated by the hydrogen gas irradiated with the light from the light source device 21.

### Second Embodiment

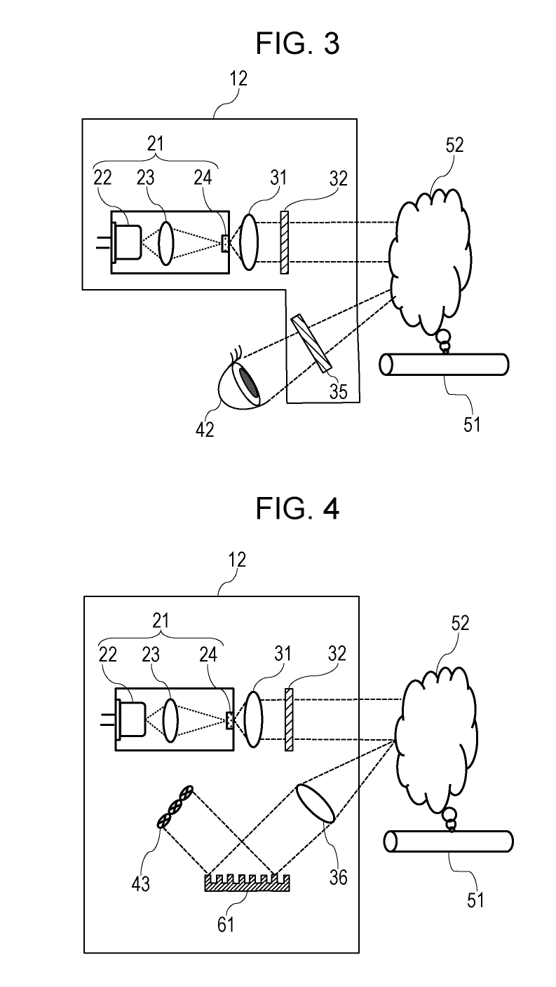

FIG. 3 illustrates an outline configuration of a device that inspects the presence or absence of hydrogen gas according to Second Embodiment of the present disclosure. An inspection device 12 includes a light source device 21, a lens 31 for irradiation, an optical bandpass filter 32 for irradiation light, and an optical bandpass filter 35 for visual check. The light source device 21 includes a semiconductor light-emitting device 22, a light collecting lens 23, and a phosphor element 24.

The light source device 21, the lens 31, and the optical bandpass filter 32 may have configurations same or similar to those in First Embodiment of the present disclosure. A peak wavelength of light of the semiconductor light-emitting device 22 can be selected as appropriate in view of a wavelength range and/or conversion efficiency of wavelength-converted light emitted from a phosphor material used in the phosphor element 24. In the present embodiment, a laser diode having a light emission wavelength of 445 nm is used as the semiconductor light-emitting device 22 so that conversion efficiency of (Y,Ga)₃Al₅O₁₂:Ce³⁺ composition used in the phosphor element 24 is maximized.

It is desirable that a FWHM of a spectrum of light with which a space to be inspected is irradiated be not more than 100 nm and not less than 10 nm. In a case where the FWHM of the irradiation light is not more than 100 nm, it is possible to easily separate Raman scattered light scattered by hydrogen gas from Raman scattered light scattered by oxygen gas or nitrogen gas in the atmosphere. Furthermore, in a case where the FWHM of the irradiation light is not less than 10 nm, it is possible to precisely detect Raman scattered light scattered by hydrogen gas.

In Second Embodiment of the present disclosure, the present or absence of hydrogen gas is visually determined. The optical bandpass filter 35 has a property of removing light in a wavelength region of the irradiation light and allowing Raman scattered light scattered by hydrogen gas to pass therethrough. Humans' relative luminosity is high for light having a wavelength of 555 nm. Accordingly, the wavelength range of the irradiation light may be selected so that a central wavelength of vibrational Stokes Raman scattered light scattered by hydrogen gas is not less than 455 nm and not more than 655 nm, more desirably not less than 485 nm and not more than 625 nm. This makes visual determination easy. Furthermore, visual determination becomes easier as a difference in color between the irradiation light and the vibrational Stokes Raman scattered light scattered by hydrogen gas becomes larger. In view of this, the wavelength range of the irradiation light may be selected so that the difference in color between the irradiation light and the vibrational Stokes Raman scattered light scattered by hydrogen gas becomes large. This makes visual determination easy for human eyes. That is, in a case where the presence or absence of hydrogen gas is visually determined as in Second Embodiment of the present disclosure, the presence or absence of hydrogen gas can be easily determined by selecting the wavelength range of the irradiation light so that the central wavelength of the vibrational Stokes Raman scattered light scattered by hydrogen gas is close to 555 nm and so that the difference in color between the irradiation light and the vibrational Stokes Raman scattered light scattered by hydrogen gas becomes large.

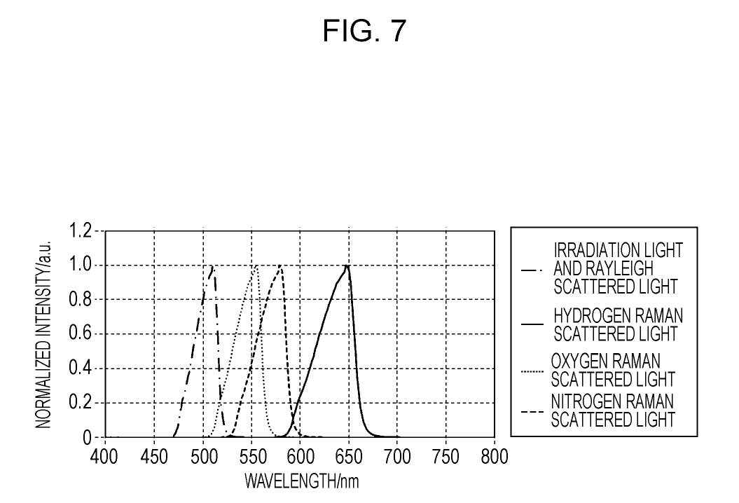

FIG. 7 illustrates spectrum shapes of Rayleigh scattered light, Raman scattered light scattered by hydrogen gas, Raman scattered light scattered by oxygen gas, and Raman scattered light scattered by nitrogen gas in a case where the wavelength region of the irradiation light is not less than 470 nm and not more than 520 nm and the FWHM of the irradiation light is 27 nm. Intensities of the spectrum shapes have been normalized. In a case where the wavelength region of the irradiation light is not less than 470 nm and not more than 520 nm (blue-green light) as illustrated in FIG. 7, it is possible to visually confirm a point to be inspected. In this case, vibrational Stokes Raman scattered light scattered by hydrogen gas has a wavelength region of not less than 584.2 nm and not more than 663.5 nm and a central wavelength of 623.4 nm. Accordingly, in a case where the optical bandpass filter 35 has a property of allowing light of not less than 584 nm to pass therethrough, the vibrational Stokes Raman scattered light scattered by hydrogen gas can be recognized as red light that is complementary to the blue-green irradiation light while minimizing the influence of oxygen gas or nitrogen gas, which is a main component in the atmosphere. This makes it possible to visually check the presence of hydrogen by human eyes.

The optical bandpass filter 35 may be a dielectric multi-layer type filter or may be an absorption type filter.

### Third Embodiment

FIG. 4 illustrates an outline configuration of a device that inspects the presence or absence of hydrogen gas and the concentration of hydrogen gas according to Third Embodiment of the present disclosure. An inspection device 13 includes a light source device 21, a lens 31 for irradiation, an optical bandpass filter 32 for irradiation light, a lens 36 for light reception, a spectrometer 61 for light reception, and a light detection device 43. The light source device 21 includes a semiconductor light-emitting device 22, a light collecting lens 23, and a phosphor element 24.

The light source device 21, the lens 31, the optical bandpass filter 32, and the lens 36 may have configurations same or similar to those in First Embodiment of the present disclosure.

In Third Embodiment of the present disclosure, light from a space 52 to be inspected is collected by the lens 36 and is then dispersed by the spectrometer 61. Thereby, the spectrometer 61 extracts the Raman scattered light of hydrogen gas from scattered light generated in the space 52 to be inspected. The type of dispersion of the spectrometer 61 is not limited to a specific one, and may be a diffraction grating type or a prism type.

The light that has been dispersed by the spectrometer 61 is detected by the light detection device 43 as a spectrum that is light intensities at respective wavelengths. The light detection device 43 may be made up of a single optical sensor or may be a multi-channel type detector made up of a plurality of optical sensors. Although an optical sensor used in the light detection device 43 is not limited to an optical sensor of a specific type, it is desirable that the optical sensor be an avalanche photodiode or a photomultiplier tube in a case where the light detection device 43 is made up of a single optical sensor. In a case where the light detection device 43 is a multi-channel type detector made up of a plurality of optical sensors, it is desirable that the plurality of optical sensors be CCD sensors or CMOS sensors.

It is possible to conduct quantitative analysis of the presence or absence and the concentration of hydrogen gas by extracting only a component attributable to Raman scattering caused by hydrogen gas from a spectrum that is output from the light detection device 43. A method for extracting only a component attributable to Raman scattering caused by hydrogen gas from a spectrum that is output from the light detection device 43 is not limited to a specific one, and can be a method such as a difference spectrum method, derivative spectrophotometry, a curve fitting method, a Fourier self-deconvolution method, or a chemometric method. The light detection device 43 may include a processing device (e.g., a microcomputer or a processor) that processes a signal from the optical sensor and a storage medium (e.g., a semiconductor memory or a hard disc) in which a processing program and processing data are stored. In this case, the processing device executes the aforementioned extraction method in accordance with the program and stores a result of the extraction in the storage medium.

Hydrogen gas detection method and device of the present disclosure make it possible to safely and precisely conduct quantitative analysis of hydrogen gas, which is colorless and odorless, in a non-contact manner and can be used for detection of leakage of hydrogen gas from a remote place in a hydrogen refueling station or a fuel cell system. Furthermore, the hydrogen gas detection device can be used as a handy hydrogen gas detection device for specifying a hydrogen leaking part of a hydrogen storage tank, a hydrogen refueling pipe, or the like.

What is claimed is:

1. A hydrogen gas inspection method comprising:
converting first light having a first wavelength to second light having a second wavelength longer than the first wavelength by using a phosphor, the first light being emitted from a semiconductor light emitting device;
irradiating a space to be inspected with the second light; and
determining whether hydrogen gas is present in the space utilizing Raman scattered light generated by the hydrogen gas irradiated with the second light.

2. The hydrogen gas inspection method according to claim 1, wherein the Raman scattered light is detected from scattered light generated in the space to be inspected.

3. The hydrogen gas inspection method according to claim 2, further comprising determining a concentration of the hydrogen gas in the space to be inspected.

4. The hydrogen gas inspection method according to claim 1, wherein
the semiconductor light-emitting device is a laser diode having a light emission peak wavelength of 360 nm to 500 nm.

5. The hydrogen gas inspection method according to claim 1, wherein
a light emission peak wavelength of the second light is 380 nm to 600 nm.

6. The hydrogen gas inspection method according to claim 1, wherein
the space to be inspected is irradiated with the second light via an optical bandpass filter, and a full width at half maximum of the second light that passes through the optical bandpass filter is 10 nm to 100 nm.

7. The hydrogen gas inspection method according to claim 1, wherein
an optical bandpass filter extracts the Raman scattered light from scattered light generated in the space to be inspected.

8. The hydrogen gas inspection method according to claim 7, wherein an optical sensor is used to detect the Raman scattered light.

9. The hydrogen gas inspection method according to claim 7, wherein
the Raman scattered light is visually checked by human eyes.

10. The hydrogen gas inspection method according to claim 1, wherein
a spectrometer extracts the Raman scattered light from scattered light generated in the space to be inspected.

11. The hydrogen gas inspection method according to claim 10, wherein an optical sensor is used to detect the Raman scattered light.

12. A hydrogen gas inspection device comprising:
a semiconductor light-emitting device that emits first light having a first wavelength;
a phosphor that converts the first light to second light having a second wavelength longer than the first wavelength and irradiates a space to be inspected with the second light; and
a light detection device that determines whether hydrogen gas is present in the space utilizing Raman scattered light generated by the hydrogen gas irradiated with the second light.
